(19) United States
(12) Patent Application Publication
(10) Pub. No.: US 2023/0295554 A1
(43) Pub. Date: Sep. 21, 2023

(54) BIOPROCESSING SYSTEM AND APPARATUS FOR REDUCING CELL SHEAR IN A BIOPROCESSING SYSTEM

(71) Applicant: GLOBAL LIFE SCIENCES SOLUTIONS USA LLC, MARLBOROUGH, MA (US)

(72) Inventors: SARAVANAN BALAKRISHNAN, BENGALURU (IN); KANDAKUMAR MURUGESAN, BENGALURU (IN); NAGARAJ RAGHAVENDRA RAO, BENGALURU (IN); MATTHEW D OUELLETTE, NEWTON, MA (US)

(73) Assignee: GLOBAL LIFE SCIENCES SOLUTIONS USA LLC, MARLBOROUGH, MA (US)

(21) Appl. No.: 18/020,527

(22) PCT Filed: Sep. 27, 2021
(86) PCT No.: PCT/EP2021/076469

§ 371 Date: Feb. 9, 2023

(30) Foreign Application Priority Data
Oct. 8, 2020 (IN) 202011043869

(51) Int. Cl. C12M 1/21; C12M 1/04; C12M 1/00

(52) U.S. Cl. CPC C12M 41/02; C12M 23/24; C12M 23/14; C12M 29/08; C12M 27/22

(57) ABSTRACT

An apparatus for managing gas bubbles in a bioprocessing system includes a body portion having a underside surface, and an opening in the body portion. The body portion is configured for placement within a bioreactor vessel such that the underside surface of the body portion is disposed in a liquid within the bioreactor vessel. The underside surface of the body portion is configured to divert rising gas bubbles in the liquid towards the opening.

### BACKGROUND

### Technical Field

Embodiments of the invention relate generally to bioprocessing systems and methods and, more particularly, to an apparatus for inhibiting or reducing cell shear caused by bubble rupture in a bioprocessing system.

### Discussion of Art

A variety of vessels, devices, components and unit operations are known for carrying out biochemical and/or biological processes and/or manipulating liquids and other products of such processes. In order to avoid the time, expense, and difficulties associated with sterilizing the vessels used in biopharmaceutical manufacturing processes, single-use or disposable bioreactor bags and single-use mixer bags are used as such vessels. For instance, biological materials (e.g., animal and plant cells) including, for example, mammalian, plant or insect cells and microbial cultures can be processed using disposable or single-use mixers and bioreactors.

Increasingly, in the biopharmaceutical industry, single use or disposable containers are used. Such containers can be flexible or collapsible plastic bags that are supported by an outer rigid structure such as a stainless steel shell or vessel. Use of sterilized disposable bags eliminates time-consuming step of cleaning of the vessel and reduces the chance of contamination. The bag may be positioned within the rigid vessel and filled with the desired fluid for mixing. An agitator assembly disposed within the bag is used to mix the fluid. Existing agitators are either top-driven (having a shaft that extends downwardly into the bag, on which one or more impellers are mounted) or bottom-driven (having an impeller disposed in the bottom of the bag that is driven by a magnetic drive system or motor positioned outside the bag and/or vessel). Most magnetic agitator systems include a rotating magnetic drive head outside of the bag and a rotating magnetic agitator (also referred to in this context as the “impeller”) within the bag. The movement of the magnetic drive head enables torque transfer and thus rotation of the magnetic agitator allowing the agitator to mix a fluid within the vessel.

Depending on the fluid being processed, the bioreactor system may include a number of fluid lines and different sensors, probes and ports coupled with the bag for monitoring, analytics, sampling, and liquid transfer. For example, a harvest port is typically located at the bottom of the disposable bag and the vessel, and allows for a harvest line to be connected to the bag for harvesting and draining of the bag. In addition, existing bioreactor systems typically utilize spargers for introducing a controlled amount of a specific gas or combination of gases into the bioreactor. A sparger outputs small gas bubbles into a liquid in order to agitate and/or dissolve the gas into the liquid, or for carbon dioxide stripping. The delivery of gas via spargers helps in mixing a substance, maintaining a homogenous environment throughout the interior of the bag, and is sometimes essential for growing cells in a bioreactor. Ideally, the spargers and the agitator are in close proximity to ensure optimal distribution of the gases throughout the container.

For cell growth in a bioreactor, oxygen is one of the most important substances that is introduced in form of dissolved oxygen through continuous sparging of air bubbles into the culture media. Typically, it is desired to sparge using smaller bubbles to achieve better mass transfer coefficient (KLa) for cell growth. However, there can be an adverse effect of the air bubbles in the bioreactor; namely, when these bubbles rise to the top surface of the liquid within the bioreactor, they burst with a high energy dissipation rate which can shear and kill cells within close proximity to the busting bubbles.

In current designs, with a larger media/air interface, bubbles burst randomly at any location with a greater number of high energy dissipation zones. Due to continuous agitation of the culture by the impeller, new cells are constantly circulated to the top of the culture, where they are exposed to the bursting sparge bubbles at the liquid/gas interface. Accordingly, this increases the number of cells that are exposed to high energy dissipation from the bursting sparge bubbles, leading to increased cell death and thus reducing the overall viable cell counts.

In view of the above, there is a need for an apparatus that is effective in reducing cell shear caused by the bursting of sparge bubbles adjacent to the liquid/gas interface, which results in increased bioreactor titers and reduced and turbidity.

### BRIEF DESCRIPTION

In an embodiment, an apparatus for reducing cell shear in a bioprocessing system is provided. The apparatus includes a body portion having a underside surface, and an opening in the body portion. The body portion is configured for placement within a bioreactor vessel such that the underside surface of the body portion is disposed in a liquid within the bioreactor vessel, and wherein the underside surface of the body portion is configured to divert rising gas bubbles in the liquid towards the opening.

In another embodiment of the invention, a bioprocessing system is provided. The bioprocessing system includes a vessel, a flexible bioprocessing bag positionable within the vessel, the flexible bioprocessing bag being configured to contain a volume of liquid, and an apparatus for reducing cell shear of cells within the liquid, the apparatus being disposed within the flexible bioprocessing bag. The apparatus includes a body portion having a underside surface, and an opening in the body portion. The underside surface of the body portion is disposed in the liquid within the flexible bioprocessing bag. The underside surface of the body portion is configured to divert rising gas bubbles in the liquid towards the opening.

In another embodiment, a method for reducing cell shear in a bioprocessing system having a bioreactor vessel containing a volume of fluid and a gas above the volume of fluid, the fluid and gas defining a fluid/gas interface, is provided. The method includes the steps of positioning an apparatus having a frusto-conical underside surface within the bioreactor vessel such that fluid within the bioreactor vessel is baffled by the underside surface of the apparatus so as to reduce a surface area of the fluid/gas interface as compared to the surface area of the fluid/gas interface in the absence of baffling of the fluid by the apparatus, and introducing a sparge gas into the volume of fluid. The underside surface of the body portion is configured to direct bubbles of the sparge gas within the fluid towards the fluid/gas interface.

In yet another embodiment, an apparatus for reducing cell shear in a bioprocessing system is provided. The apparatus includes a body having a frusto-conical underside surface, and a central opening in the body portion. The body is positionable around an upper end of a flexible bioprocessing bag such that the upper end of the flexible bioprocessing bag extends through the central opening. The frusto-conical underside surface baffles a liquid within the flexible bioprocessing bag so as to reduce a surface area of a liquid/gas interface as compared to the surface area of the liquid/gas interface in the absence of the apparatus, and the underside surface of the body is configured to direct gas bubbles within the fluid towards the opening and to the liquid/gas interface.

### DRAWINGS

The present invention will be better understood from reading the following description of non-limiting embodiments, with reference to the attached drawings, wherein below:

FIG. 1 is a front elevational view of a bioreactor system according to an embodiment of the invention.

FIG. 2 is a simplified side elevational, cross-sectional view of the bioreactor system of FIG. 1.

FIG. 3 is a simplified side elevational, cross-sectional view of the bioreactor system of FIG. 1, illustrating an apparatus for reducing cell shear according to an embodiment of the present invention.

FIG. 4 is a side elevational view of the apparatus for reducing cell shear.

FIG. 5 is an enlarged, cross-sectional view of area A of FIG. 3, illustrating the isolation chamber of the apparatus.

FIG. 6 is a simplified schematic illustration of a bioprocessing system having an apparatus for reducing cell shear according to another embodiment of the invention.

FIG. 7 is a simplified schematic illustration of a bioprocessing system having an apparatus for reducing cell shear according to another embodiment of the invention.

### DETAILED DESCRIPTION

Reference will be made below in detail to exemplary embodiments of the invention, examples of which are illustrated in the accompanying drawings. Wherever possible, the same reference characters used throughout the drawings refer to the same or like parts.

As used herein, the term “flexible” or “collapsible” refers to a structure or material that is pliable, or capable of being bent without breaking, and may also refer to a material that is compressible or expandable. An example of a flexible structure is a bag formed of polyethylene film. The terms “rigid” and “semi-rigid” are used herein interchangeably to describe structures that are “non-collapsible,” that is to say structures that do not fold, collapse, or otherwise deform under normal forces to substantially reduce their elongate dimension. Depending on the context, “semi-rigid” can also denote a structure that is more flexible than a “rigid” element, e.g., a bendable tube or conduit, but still one that does not collapse longitudinally under normal conditions and forces.

A “vessel,” as the term is used herein, means a flexible bag, a flexible container, a semi-rigid container, or a rigid container, as the case may be. The term “vessel” as used herein is intended to encompass bioreactor vessels having a wall or a portion of a wall that is flexible or semi-rigid, single use flexible bags, as well as other containers or conduits commonly used in biological or biochemical processing, including, for example, cell culture/purification systems, mixing systems, media/buffer preparation systems, and filtration/purification systems. As used herein, the term “bag” means a flexible or semi-rigid container or vessel used, for example, as a bioreactor or mixer for the contents within.

Embodiments of the invention provide apparatuses and devices for reducing cell shear in a bioprocessing system. In one embodiment, an apparatus includes a body portion having a underside surface, and an opening in the body portion. The body portion is configured for placement within a bioreactor vessel such that the underside surface of the body portion is disposed in a liquid within the bioreactor vessel, and such that a portion the apparatus extends above a liquid/gas interface of the bioprocessing system. When the apparatus is disposed in the bioreactor vessel, a surface area of the liquid/gas interface is reduced by the apparatus as compared to the surface area of the liquid/gas interface of the bioprocessing system in the absence of the apparatus. The underside surface of the body portion is configured to direct gas bubbles within the liquid towards the opening and to the liquid/gas interface

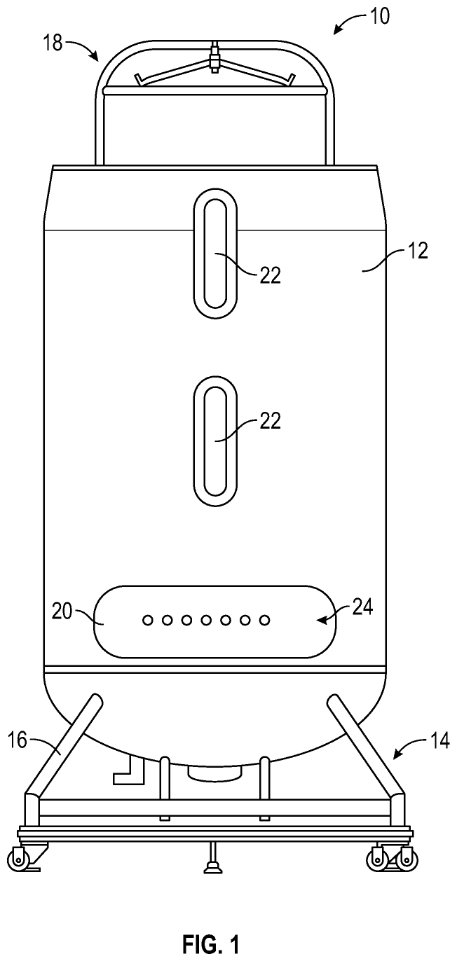

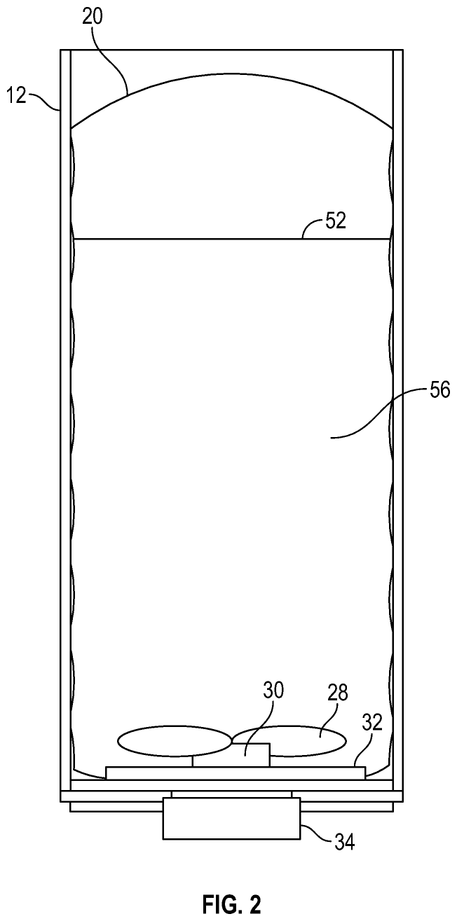

With reference to FIGS. 1 and 2, a bioreactor system 10 according to an embodiment of the invention is illustrated. The bioreactor system 10 includes a generally rigid bioreactor vessel or support structure 12 mounted atop a base 14 having a plurality of legs 16. The vessel 12 may be formed, for example, from stainless steel, polymers, composites, glass, or other metals, and may be cylindrical in shape, although other shapes may also be utilized without departing from the broader aspects of the invention. The vessel 12 may be outfitted with a lift assembly 18 that provides support to a single-use, flexible bag 20 disposed within the vessel 12. The vessel 12 can be any shape or size as long as it is capable of supporting a single-use flexible bioreactor bag 20. For example, according to one embodiment of the invention the vessel 12 is capable of accepting and supporting a 10-2000 L flexible or collapsible bioprocess bag assembly 20.

The vessel 12 may include one or more sight windows 22, which allows one to view a fluid level within the flexible bag 20, as well as a window 24 positioned at a lower area of the vessel 12. The window 24 allows access to the interior of the vessel 12 for insertion and positioning of various sensors and probes (not shown) within the flexible bag 20, and for connecting one or more fluid lines to the flexible bag 20 for fluids, gases, and the like, to be added or withdrawn from the flexible bag 20. Sensors/probes and controls for monitoring and controlling important process parameters include any one or more, and combinations of: temperature, pressure, pH, dissolved oxygen (DO), dissolved carbon dioxide (pCO₂), mixing rate, and gas flow rate, for example.

With specific reference to FIG. 2, a schematic side elevational, cutaway view of the bioreactor system 10 is illustrated. As shown therein, the single-use, flexible bag 20 is disposed within the vessel 12 and restrained thereby. In embodiments, the single-use, flexible bag 20 is formed of a suitable flexible material, such as a homopolymer or a copolymer. The flexible material can be one that is USP Class VI certified, for example, silicone, polycarbonate, polyethylene, and polypropylene. Non-limiting examples of flexible materials include polymers such as polyethylene (for example, linear low density polyethylene and ultra-low density polyethylene), polypropylene, polyvinylchloride, polyvinyldichloride, polyvinylidene chloride, ethylene vinyl acetate, polycarbonate, polymethacrylate, polyvinyl alcohol, nylon, silicone rubber, other synthetic rubbers and/or plastics. In an embodiment, the flexible material may be a laminate of several different materials such as, for example Fortem™ Bioclear™ 10 and Bioclear 11 laminates, available from GE Healthcare Life Sciences. Portions of the flexible container can comprise a substantially rigid material such as a rigid polymer, for example, high density polyethylene, metal, or glass. The flexible bag may be supplied pre-sterilized, such as using gamma irradiation.

The flexible bag 20 contains an impeller 28 attached to a magnetic hub 30 at the bottom center of the inside of the bag, which rotates on an impeller plate (not shown) also positioned on the inside bottom of the bag 20. Together, the impeller 28 and hub 30 (and in some embodiments, the impeller plate) form an impeller assembly. A magnetic drive 34 external to the vessel 12 provides the motive force for rotating the magnetic hub 30 and impeller 28 to mix the contents of the flexible bag 20. While FIG. 2 illustrates the use of a magnetically-driven impeller, other types of impellers and drive systems are also possible, including top-driven impellers.

As also illustrated in FIG. 2, the flexible bag 20 contains a sparger device 32 that can be engaged with a port (not shown) on the bottom of the flexible bag 20, which receives a supply of gas from a gas supply line. The sparger device 32, as is known in the art, is configured to introduce gas bubbles into the culture/media within the flexible bag 20. While FIGS. 1 and 2 illustrate the bioreactor vessel 12 as containing a single use, flexible bag 20, it is contemplated that the flexible bag 20 may be omitted in which case the bioprocessing/culturing operations can take place directly within the vessel 12.

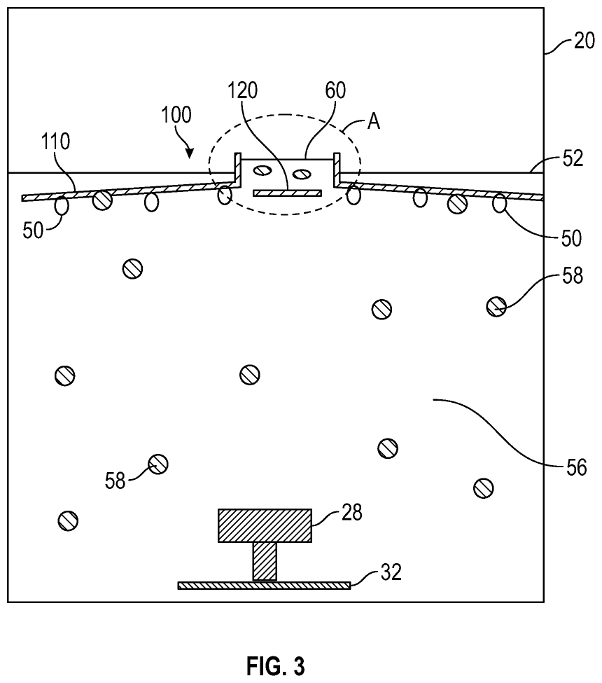

With reference to FIG. 3, the bioreactor/bioprocessing system 10 of the invention further includes an apparatus 100 that reduces and/or eliminates cell shear that may typically result from the bursting of sparge gas bubbles 50 at the liquid/gas interface 52 (shown in FIG. 2). As indicated above, while the use of sparging is important for cell growth, the gas bubbles 50 produced by the sparger 32 rise from the sparger 32, through the culture media/liquid 56, to the liquid/gas interface 52 where they burst. This bursting of the bubbles 50 releases energy that can kill cells 58 suspended in the media 56 within a certain proximity to the bursting bubbles 50. As shown in FIG. 3, the apparatus 100 is configured as a separator that is configured to float atop the culture media/liquid 56 within the bioprocessing bag 20, at the liquid/gas interface 52, for the purposes disclosed hereinafter. Accordingly, in an embodiment, the apparatus 100, including the body portion 110 thereof, may be formed from a material that is less dense than the culture media/liquid 56 such as, for example, plastic. In an embodiment, the body portion 110 is substantially rigid.

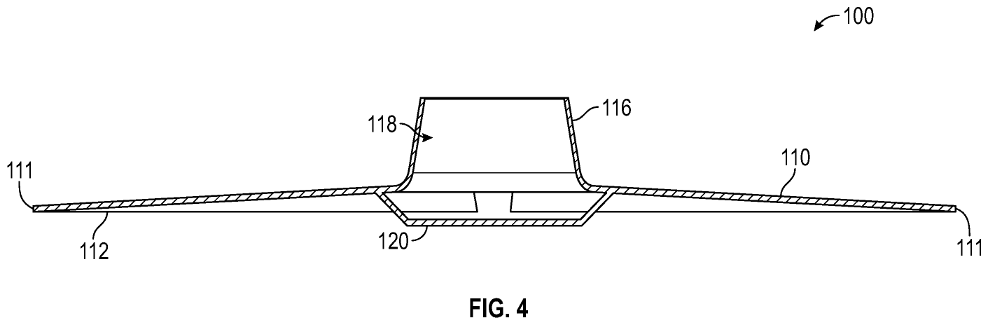

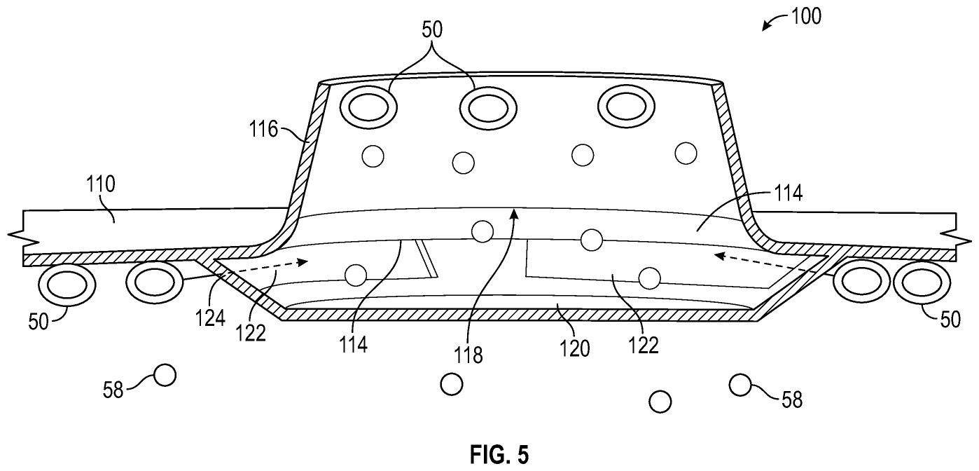

With specific reference to FIGS. 4 and 5, in an embodiment, the apparatus 100 includes a generally disc-shaped body portion 110 having a underside surface 112, and at least one opening 114 in the body portion 110. The peripheral shape and size of the body portion 110 closely corresponds to the cross-sectional shape and size of the bioreactor vessel 10 within which the apparatus 100 is intended to be positioned (such that a substantial entirety of the cross-sectional area of the bioreactor vessel 12 is occupied by the apparatus 100). In an embodiment, the opening 114 is centrally located with respect to peripheral edges 111 of the body portion 110. A peripheral wall 116 extends upwardly from the body portion 110 and generally circumscribes the opening 114, forming a tubular or frusto-conical isolation chamber 118. As shown in FIGS. 3 and 4, the isolation chamber 118 is open at the top, however, it is also envisioned that the chamber 118 may be closed at the top.

In an embodiment, the underside surface 112 of the body portion 110 has a tapered, frusto-conical or similar shape/configuration, such that the outer, peripheral edges 111 of the body portion 110 are at a lower vertically-spaced location than the opening 114. As discussed hereinafter, this configuration helps direct rising sparge gas bubbles 50 toward the opening 110. With further reference to FIGS. 4 and 5, the apparatus further includes an isolation plate 120 that extends across the opening 114 and is vertically spaced from the opening (i.e., it is positioned below the opening 114). The isolation plate 120 has at least one, and preferably a plurality of, slots, apertures or passages 122 that allow for fluid communication between the culture media 56 within the bioprocessing bag 20 and the isolation chamber 118. As shown in FIG. 4, the slots 122 are radial slots (facing generally outward), upper edges of which are located in close proximity to the underside surface 112 of the body portion 110 so that bubbles traveling along the underside surface 112 of the body portion 110 can pass easily into the isolation chamber 118 through the slots 122. In an embodiment, the isolation plate 120 is generally disc shaped (having a short, cylindrical or frusto-conical sidewall 124 that joins the isolation plate 120 to the body portion 100, and wherein the slots 122 are formed in the sidewall 124.

Referring back to FIG. 3, the apparatus 100 is configured for placement within the bioreactor vessel 12 (and, in particular, within the flexible bioprocessing bag 20, where utilized) such that the underside surface 112 of the body portion 100 is disposed in the liquid/media 56 within the bioreactor vessel 12, and such that a portion of the peripheral sidewall 116 extends above the liquid/gas interface 52 of the bioprocessing system 10. In such position, as shown in FIG. 3, the apparatus 100 functions to reduce the surface area of the liquid/gas interface that is seen by the cells 58 and bubbles 50 as compared to the surface area of the liquid/gas interface in the absence of the apparatus 100.

As alluded to above, in operation, as the sparge gas bubbles 50 rise upwardly through the media 58 within the bag 20, they contact the underside surface 112 of the body portion 110 of the apparatus 100 and travel along the underside surface 112 toward the opening 114 as a result of the tapered or angled configuration of the underside surface 112. The bubbles 50 enter the isolation chamber 118 through the slots 122 and the opening 114, as indicated by the arrows in FIG. 5, where they are permitted to reach the much smaller liquid/gas interface 60 within the isolation chamber 118. Rather than bursting across the entire cross-sectional area of the bioreactor vessel 10, the bursting bubbles are, with the apparatus 100, sequestered in the isolation chamber 118 where their detrimental effects on the cells are minimized.

The apparatus 100 of the invention, therefore, serves to reduce cell shear and cell deaths by bubble bursting in at least two ways. First, by reducing the surface area of the liquid/gas interface, as disclosed above, the total cell deaths that occur due to bubble rupture can be proportionally reduced (as the zone/area of high energy dissipation rate to which the cells are exposed is reduced). In particular, as most bubbles 50 are isolated within the isolation chamber 118 (where they are permitted to burst at the reduced liquid/gas interface 60), the energy released upon bursting is likewise isolated to the small, confined area of the isolation chamber 118 where it does not adversely impact any of the cells outside the isolation chamber 118. The configuration of the apparatus 100 also serves to isolate the small isolation chamber 118, where the bubbles are permitted to burst, from the rest of the processing volume of the bag 20, so that there is very little, if any, active mixing or exchange of cells or media between the main processing volume and the isolation chamber 118. As a result, few cells are placed in close proximity to the busting gas bubbles. Liquid mixing simulations have demonstrated that by isolating a small volume by a means of mechanical separation (i.e., by using the apparatus 100), mixing of cells from main stream volume to isolation chamber 118 can be prevented, which effectively reduces the number of new cells that are exposed to the high energy dissipation rate of bursting bubbles in the isolation chamber, thereby increasing cell viability.

Second, the inverted cone or funnel-shaped body portion 110 forces the bubbles 50 to coalesce prior to reaching the gas/liquid interface 60 within the isolation chamber 118. As will be appreciated, purposefully increasing bubble size via coalescence serves to reduce the energy dissipation rate that occurs during bubble rupture to sub-lethal levels, as increased bubble size has a lower energy dissipation rate. Accordingly, even for cells present within the isolation chamber 118, the energy dissipation rate of the larger bubbles may not be sufficient to cause significant cell death. In connection with the above, in an embodiment, the underside surface 112 of the body portion may include a plurality of grooves (not shown)that are oriented and positioned so as to facilitate coalescence of the gas bubbles 50 as they travel along the underside surface 112 towards the opening 114.

By reducing or eliminating cell death from bubble rupture, bioreactor cell densities and titers can be increased and turbidity reduced. The apparatus of the invention may also reduce or eliminate the amount of poloxamer (e.g., Puronic) used during bioprocessing operations, which results in fewer process steps required, and reduces Pluronic induced foaming. Still further, reducing turbidity generated by cell death may also enable better perfusion reactor performance.

In connection with the above, the apparatus of the invention not only reduces or eliminates cell death due to bubble rupture, but also minimizes or reduces foaming within the bioreactor vessel. Small bubbles, such as those generated during sparging, tend to pack very closely together, forming a close, well-established foam which is difficult to disrupt, while larger bubbles form a loose foam which bursts more effectively and is, thus, easier to control. As discussed above, the apparatus of the invention facilitates coalescence, creating large gas bubbles which can more easily be controlled and/or eliminated. This is particularly true where the large bubbles formed by coalescence are diverted and collected in the isolation chamber 118. In an embodiment, it is further contemplated that an anti-foam device may be positioned above or within the isolation chamber 118 for dispensing anti-foam therein. Thus, the dispensing of anti-foam may only be necessary in the small confined area of the isolation chamber (and the reduced liquid/gas interface) rather than the entire liquid/gas interface within the bioreactor vessel.

While FIGS. 3-5 illustrate the opening 114 as being a single opening that is centrally located on the body portion 110, the invention is not intended to be so limited in this regard. In particular, it is contemplated that the opening 114 may be offset from the center of the body portion 110, and that more than one opening and/or isolation chamber may be present. In any such case, the underside surface 112 of the body portion 110 is shaped and configured so as to collect the rising gas bubbles and to guide or direct them towards the opening(s) and isolation chamber(s).

It is contemplated that the apparatus 100 may float atop the process volume and thus, in this regard, may self-adjust to the volume of liquid within the bioprocessing system 20. Alternatively, or in addition, the apparatus 100 may be raised or lowered during the bioprocessing operation(s) to accommodate different bioreactor fill volumes. Advantageously, the shape of the apparatus 100 is also such that it can be included in the bioprocessing bag during storage and transport, in which case it can be received atop the impeller 28, protecting the same from damage.

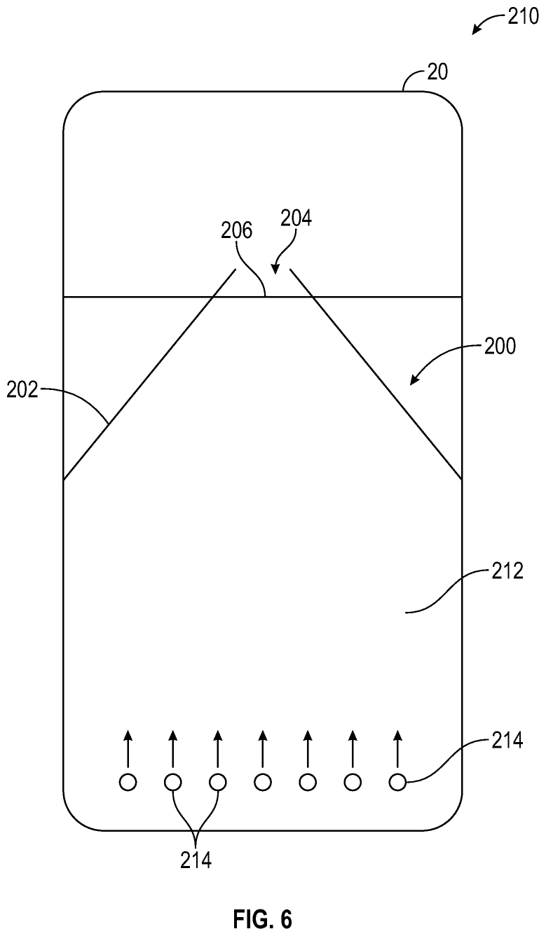

Turning now to FIG. 6, an apparatus 200 for reducing and/or eliminating cell shear according to another embodiment of the present invention is illustrated as deployed in a bioprocessing system 210 having a flexible bioprocessing bag 20 (and/or rigid vessel) defining a processing volume. As illustrated therein, the apparatus 200 has a body 202 in the shape of an inverted cone, and a centrally located opening 204. The apparatus 200 is configured as a separator that is configured to float atop the process volume 212 such that the liquid/gas interface 206 is reduced in size as compared to the liquid/gas interface of the bioprocessing system 210 in the absence of the apparatus. Similar to the apparatus 100 disclosed above, the apparatus 200 is configured to receive the bubbles 214 as they rise toward the top surface of the process volume 212, and to direct the bubbles 214 towards the opening 204 and the reduced liquid/gas interface 206 where they burst. Like apparatus 100, this serves to reduce cell shear and cell deaths by bubble bursting by reducing the surface area of the liquid/gas interface (i.e., reducing the area of the zone of high energy dissipation rate due to bubble bursting), and increases bubble coalescence. Similar to the embodiment described above, the underside surface of the body portion 202 may be grooved to facilitate coalescence. In addition, like the apparatus 100, the inverted cone shape of the apparatus 200 can be utilized to help protect the impeller during storage and transport.

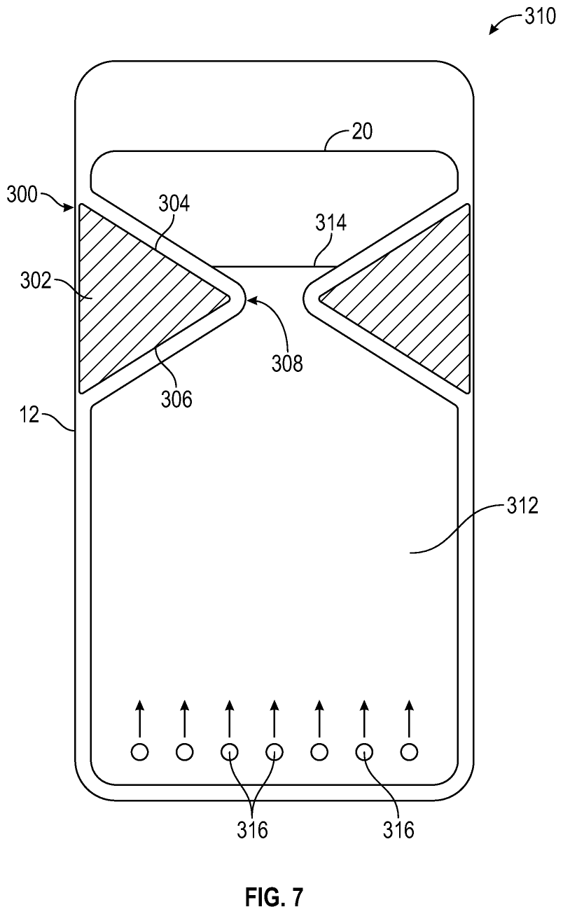

Turning finally to FIG. 7, an apparatus 300 for reducing and/or eliminating cell shear according to another embodiment of the present invention is illustrated as deployed in a bioprocessing system 310 having a rigid bioreactor vessel 12 supporting a flexible bioprocessing bag 20 containing a process volume 312. As illustrated therein, the apparatus 300 includes a body 302 that is generally wedge shape in cross section, such that an upward-facing surface 304 is in the shape of a funnel, and a downward-facing surface 306 is in the shape of an inverted funnel. The apparatus 300 also includes a centrally located opening 308 forming a passage through the body 302.

As shown in FIG. 7, an upper portion of the flexible bioprocessing bag 20 is received through the opening 308, such that the body 302 baffles the fluid within the bag 20. As shown, this functions to reduce the area of the gas/liquid interface 314. In an embodiment, the vertical position of the apparatus 300 with respect to the bioprocessing bag 20 may be adjusted so as to selectively control the area of the interface 314. In particular, lowering the apparatus 300 within the vessel 12 will increase the area of the gas/liquid interface 314, while raising the apparatus 300 within the vessel 12 will decrease the area of thee gas/liquid interface 314 (so long as the apparatus 300 is in a position where it baffles the fluid within the bag 20).

Similar to the embodiments disclosed above, the apparatus 300 is configured to direct the rising sparge gas bubbles 316 within the process volume 312 toward the opening 308 and the reduced liquid/gas interface 314 where they burst. As disclosed, this serves to reduce cell shear and cell deaths by bubble bursting by reducing the surface area of the liquid/gas interface (i.e., reducing the area of the zone of high energy dissipation rate due to bubble bursting), and increasing bubble coalescence.

Notably, the apparatus 300 is not disposed within the bag 20, so that it is not in contact with the process volume 312 (although it baffles the process volume by baffling the bag 20). In an embodiment, the apparatus 300 may be integrated with the interior sidewall of the vessel 12. For example, the apparatus 300 may be fixedly secured or unitarily formed with the interior sidewalls of the vessel, or it may be connected to the vessel 12 and selectively positionable at various vertical locations.

In an embodiment, an apparatus for reducing cell shear in a bioprocessing system is provided. The apparatus includes a body portion having a underside surface, and an opening in the body portion. The body portion is configured for placement within a bioreactor vessel such that the underside surface of the body portion is disposed in a liquid within the bioreactor vessel, and wherein the underside surface of the body portion is configured to divert rising gas bubbles in the liquid towards the opening. In an embodiment, the body portion is configured such that a portion of the apparatus extends above a liquid/gas interface of the bioprocessing system. When the apparatus is disposed in the bioreactor vessel, a surface area of the liquid/gas interface is reduced by the apparatus as compared to the surface area of the liquid/gas interface of the bioprocessing system in the absence of the apparatus. The underside surface of the body portion is configured to direct gas bubbles within the liquid towards the opening and to the liquid/gas interface. In an embodiment, the underside surface has a tapered shape, configured to direct the gas bubbles towards the opening and to the liquid/gas interface. In an embodiment, the underside surface has a frusto-conical shape. In an embodiment, the underside surface has a plurality of grooves configured to facilitate coalescence of the gas bubbles. In an embodiment, the apparatus further includes a peripheral wall extending upwardly from the body portion and circumscribing the opening, the peripheral wall defining an isolation chamber therein, wherein the liquid/gas interface is located within the isolation chamber above the opening. In an embodiment, the isolation chamber has a top opening. In an embodiment, the apparatus includes an isolation plate extending across the opening, the isolation plate having at least one aperture allowing for fluid communication with the isolation chamber through the at least one opening. In an embodiment, the body portion is formed from plastic.

According to another embodiment of the invention, a bioprocessing system is provided. The bioprocessing system includes a vessel, a flexible bioprocessing bag positionable within the vessel, the flexible bioprocessing bag being configured to contain a volume of liquid, and an apparatus for reducing cell shear of cells within the liquid, the apparatus being disposed within the flexible bioprocessing bag. The apparatus includes a body portion having a underside surface, and an opening in the body portion. The underside surface of the body portion is disposed in the liquid within the flexible bioprocessing bag such that a portion the body portion extends above a liquid/gas interface. When the apparatus is disposed in the liquid, a surface area of the liquid/gas interface is reduced by the apparatus as compared to the surface area of the liquid/gas interface in the absence of the apparatus. The underside surface of the body portion is configured to direct gas bubbles within the liquid towards the opening and to the liquid/gas interface. In an embodiment, the underside surface of the body portion of the apparatus has a tapered shape, configured to direct the gas bubbles towards the opening and to the liquid/gas interface. In an embodiment, the underside surface has a frusto-conical shape. In an embodiment, the underside surface has a plurality of grooves configured to facilitate coalescence of the gas bubbles. In an embodiment, the apparatus further includes a peripheral wall extending upwardly from the body portion and circumscribing the opening, the peripheral wall defining an isolation chamber therein, wherein the liquid/gas interface is located within the isolation chamber above the opening. In an embodiment, the isolation chamber has a top opening. In an embodiment, the apparatus includes an isolation plate extending across the opening, the isolation plate having at least one aperture allowing for fluid communication with the isolation chamber through the at least one aperture.

In another embodiment, a method for reducing cell shear in a bioprocessing system having a bioreactor vessel containing a volume of fluid and a gas above the volume of fluid, the fluid and gas defining a fluid/gas interface, is provided. The method includes the steps of positioning an apparatus having a frusto-conical underside surface within the bioreactor vessel such that fluid within the bioreactor vessel is baffled by the underside surface of the apparatus so as to reduce a surface area of the fluid/gas interface as compared to the surface area of the fluid/gas interface in the absence of baffling of the fluid by the apparatus, and introducing a sparge gas into the volume of fluid. The underside surface of the body portion is configured to direct bubbles of the sparge gas within the fluid towards the fluid/gas interface. In an embodiment, the apparatus further includes a body portion having the underside surface, a central opening and peripheral wall extending upwardly from the body portion and circumscribing the central opening, the peripheral wall defining an isolation chamber therein, wherein the method includes positioning the apparatus such that fluid/gas interface is located within the isolation chamber above the opening. In an embodiment, the apparatus further includes an isolation plate extending across the opening, the isolation plate having at least one aperture allowing for fluid communication with the isolation chamber through the at least one aperture. In an embodiment, the method further includes the step of moving the apparatus from a first position where the apparatus covers and protects an impeller of the bioprocessing system, to a second position where the fluid within the bioreactor vessel is baffled by the underside surface of the apparatus.

In yet another embodiment, an apparatus for reducing cell shear in a bioprocessing system is provided. The apparatus includes a body having a frusto-conical underside surface, and a central opening in the body portion. The body is positionable around an upper end of a flexible bioprocessing bag such that the upper end of the flexible bioprocessing bag extends through the central opening. The frusto-conical underside surface baffles a liquid within the flexible bioprocessing bag so as to reduce a surface area of a liquid/gas interface as compared to the surface area of the liquid/gas interface in the absence of the apparatus, and the underside surface of the body is configured to direct gas bubbles within the fluid towards the opening and to the liquid/gas interface. In an embodiment, the apparatus is positionable at varying vertical locations within the bioprocessing system. In an embodiment, the apparatus is connected to inner sidewall surface of a bioreactor vessel that receives the flexible bioprocessing bag.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property.

This written description uses examples to disclose several embodiments of the invention, including the best mode, and also to enable one of ordinary skill in the art to practice the embodiments of invention, including making and using any devices or systems and performing any incorporated methods. The patentable scope of the invention is defined by the claims, and may include other examples that occur to one of ordinary skill in the art. Such other examples are intended to be within the scope of the claims if they have structural elements that do not differ from the literal language of the claims, or if they include equivalent structural elements with insubstantial differences from the literal languages of the claims.

1. An apparatus for managing gas bubbles in a bioprocessing system, comprising:
a body portion having an underside surface; and
an opening in the body portion;
wherein the body portion is configured for placement within a bioreactor vessel such that the underside surface of the body portion is disposed in a liquid within the bioreactor vessel; and
wherein the underside surface of the body portion is configured to divert rising gas bubbles in the liquid towards the opening.

2. The apparatus of claim 1, wherein:
a portion of the apparatus extends above a liquid/gas interface within the bioreactor vessel;
wherein when the apparatus is disposed in the bioreactor vessel, a surface area of the liquid/gas interface accessible to the gas bubbles via the opening is reduced by the apparatus as compared to the surface area of the liquid/gas interface of the bioprocessing system accessible to the gas bubbles in the absence of the apparatus; and
wherein the underside surface of the body portion is configured to direct gas bubbles within the liquid towards the opening and to the liquid/gas interface accessible via the opening.

3. The apparatus of claim 1, wherein:
the underside surface has a tapered shape, configured to direct the gas bubbles towards the opening and to the liquid/gas interface.

4. The apparatus of claim 1, wherein:
the underside surface has a frusto-conical shape.

5. The apparatus of claim 1, wherein:
the underside surface has a plurality of grooves configured to facilitate coalescence of the gas bubbles.

6. The apparatus of claim 1, further comprising:
a peripheral wall extending upwardly from the body portion and circumscribing the opening, the peripheral wall defining an isolation chamber therein;
wherein the liquid/gas interface accessible to the gas bubbles is located within the isolation chamber above the opening.

7. The apparatus of claim 6, wherein:
the isolation chamber has a top opening.

8. The apparatus of claim 6, further comprising:
an isolation plate extending across the opening, the isolation plate having at least one aperture allowing for fluid communication with the isolation chamber through the at least one opening.

9. A bioprocessing system, comprising:
a vessel;
a flexible bioprocessing bag positionable within the vessel, the flexible bioprocessing bag being configured to contain a volume of liquid; and
an apparatus for reducing cell shear of cells within the liquid and/or for reducing foaming, the apparatus being disposed within the flexible bioprocessing bag and including:
a body portion having a underside surface; and
an opening in the body portion;
wherein the underside surface of the body portion is configured to divert rising gas bubbles within the liquid towards the opening.

10. The bioprocessing system of claim 9, wherein:
the apparatus is disposed in the liquid within the flexible bioprocessing bag such that a portion the body portion extends above a liquid/gas interface;
wherein when the apparatus is disposed in the liquid a portion the apparatus extends above a liquid/gas interface within the bioreactor vessel;
wherein when the apparatus is disposed in the bioreactor vessel, a surface area of the liquid/gas interface accessible to the gas bubbles via the opening is reduced by the apparatus as compared to the surface area of the liquid/gas interface of the bioprocessing system accessible to the gas bubbles in the absence of the apparatus; and
wherein the underside surface of the body portion is configured to direct gas bubbles within the liquid towards the opening and to the liquid/ gas interface accessible via the opening.

11. The bioprocessing system of claim 9, wherein:
the underside surface of the body portion of the apparatus has a tapered shape, configured to direct the gas bubbles towards the opening and to the liquid/gas interface.

12. The bioprocessing system of claim 9, wherein:
the underside surface has a frusto-conical shape.

13. The bioprocessing system of claim 9, wherein:
the underside surface has a plurality of grooves configured to facilitate coalescence of the gas bubbles.

14. The bioprocessing system of claim 9, wherein:
the apparatus further includes a peripheral wall extending upwardly from the body portion and circumscribing the opening, the peripheral wall defining an isolation chamber therein;
wherein the liquid/gas interface accessible to the gas bubbles is located within the isolation chamber above the opening

15. The bioprocessing system of claim 13, wherein:
the isolation chamber has a top opening.
